(19) United States
(12) Patent Application Publication
(10) Pub. No.: US 2017/0304159 A1
(43) Pub. Date: Oct. 26, 2017

(54) COMPONENTS FOR SKIN CARE COMPOSITIONS AND THEIR USE, METHODS FOR IMPROVING SKIN CARE COMPOSITIONS

(71) Applicant: Imerys Minerals Limited, Par Cornwall (GB)

(72) Inventors: Agnes FIOLET, Toulouse (FR); Philippe MAILLARD, Tournefeuille (FR); Gilles MELI, La Fauga (FR); Laure PAGIS, Toulouse (FR)

(21) Appl. No.: 15/517,104

(22) PCT Filed: Oct. 7, 2015
(86) PCT No.: PCT/EP2015/073143

§ 371 Date: Apr. 5, 2017

(30) Foreign Application Priority Data
Oct. 7, 2014 (EP) 14290303.8

(51) Int. Cl. A61K 8/02; A61K 8/27; A61K 8/29; A61Q 17/04

(52) U.S. Cl. CPC A61K 8/0254; A61Q 17/04; A61K 8/27; A61K 8/29; A61K 2800/651

(57) ABSTRACT

The present invention relates to skin care compositions comprising one or more inorganic particulate materials selected from the group consisting of delaminated talc, delaminated bentonite, an alkaline earth metal carbonate or sulphate, such as calcium carbonate, magnesium carbonate, dolomite, gypsum, a hydrous kandite clay such as delaminated kaolin, halloysite or ball clay, an anhydrous or calcined kandite clay such as metakaolin or fully calcined kaolin, mica, perlite or diatomaceous earth, or magnesium hydroxide, or aluminium trihydrate, or combinations thereof; and/for wherein the inorganic particulate material has a BET surface area of least about 10.0 m²/g. The present invention further extends to the use of such compositions.

### FIELD OF THE INVENTION

The present invention relates to skin care compositions with improved organoleptic characteristics and the use of inorganic particulate materials as components of such compositions. According to the present invention, improved skin care compositions are provided, wherein the inorganic particulate material is used as an inorganic extender, such as for example a UV-filter extender. Also according to the present invention, methods for providing improved skin care compositions are provided.

### BACKGROUND OF THE INVENTION

Skin care compositions, such as for example sun care compositions (such as sunscreens or sun creams), day creams, body lotions, shower gels, shampoos, foundations, or baby creams, may be present in the form of water-in-oil emulsions, oil-in-water emulsions, or emulsified gels.

Sunscreens or sun creams are lotions, sprays, gels or other topical products that absorb or reflect some of the sun's ultraviolet (UV) radiation on the skin exposed to sunlight and thus help protect against sun damage. State of the art sunscreens may be in the form of water-in-oil or oil-in-water emulsions comprising inorganic and/or organic UV-filters. Other skin care compositions may also have sun care properties, imparted for example by inorganic and/or organic UV-filters.

Common inorganic UV-filters include zinc oxide (ZnO) and titanium dioxide (TiO₂), which may act both as mechanical sun blockers and as UV-filters in sunscreens. Due to their high opacity and refractive index, ZnO and TiO₂ have the drawbacks of a tendency to cause white streaks after application. They may also induce photocatalysis and cause skin alterations when applied to the skin. Furthermore, TiO₂ is an expensive raw material and increases the cost of finished sunscreen products.

The use of talc-based additives as carriers or additives in sunscreen compositions has previously been described.

US 2012/0082708 A1 discloses sunscreen agents comprising up to 20 wt.-% of a composite powder obtained by treating talc with methylene bis-benzotriazolyl tetramethylbutylphenol (an organic UV-filter) and dimethicone, and 8 wt.-% titanium dioxide. The process for coating the talc particles can be cumbersome and expensive.

U.S. Pat. No. 6,159,481 discloses high SPF (sun protection factor) compositions comprising up to 2.5 wt.-% micronized talc besides titanium dioxide, iron oxide, zinc oxide and a selenium yeast complex as a combined SPF-enhancer and skin protection agent. The compositions have complex component combinations and can be cumbersome and expensive to prepare.

EP 2 692 338 A1 discloses water-in-oil emulsions as sunscreen compositions comprising hydrophobicized plate-like powders, which is a silicone-coated plate-like powder, such as for example a silicone-coated talc. It is unclear what the effect of the hydrophobicized plate-like powders in the composition is, if any, and their preparation is complex.

Furthermore, it is a common need in all skin care compositions to provide improved organoleptic properties, such as improved visual appearance and tactile sensation of a composition when applied to the human skin, or ease of application (applicability), sensation on touching the skin, or consistency, softness or stickiness, or the appearance of streaking as unwanted white lines on the skin after application.

For example, US 2012/0021027 A1 discloses a film-forming cosmetic composition providing improved feel, look and wear characteristics. The compositions have complex component combinations and can be cumbersome and expensive to prepare.

The state of the art therefore constitutes a problem.

### SHORT DESCRIPTION OF THE INVENTION

The present invention is defined in the appended claims.

In one embodiment, the present invention discloses a skin care composition comprising one or more inorganic particulate materials, selected from the group consisting of delaminated talc, delaminated bentonite, an alkaline earth metal carbonate or sulphate (such as calcium carbonate, magnesium carbonate, dolomite, or gypsum), a hydrous kandite clay (such as delaminated kaolin, halloysite or ball clay), an anhydrous or calcined kandite clay (such as metakaolin or fully calcined kaolin, wollastonite, delaminated mica, perlite or diatomaceous earth), or magnesium hydroxide, or aluminium trihydrate, or combinations thereof; and/or the inorganic particulate material has a BET surface area of least about 10.0 m²/g. A skin care composition according to this invention has improved organoleptic compositions and, if required, good SPF.

In one embodiment, the inorganic particulate material may be selected from the group consisting of delaminated talc, or an alkaline earth metal carbonate or sulphate (such as calcium carbonate, magnesium carbonate, dolomite, gypsum), or halloysite, or ball clay, or metakaolin, or wollastonite, or perlite or diatomaceous earth, or magnesium hydroxide, or aluminium trihydrate, or combinations thereof. In one embodiment, the inorganic particulate material comprises one or more of delaminated talc, diatomaceous earth and perlite. It was found that such materials provided the advantages of the present invention as particularly strong improvements. For example, the inorganic particulate material may be selected from just one of delaminated talc, diatomaceous earth and perlite.

According to one embodiment of the present invention the inorganic particulate material may be a combination of delaminated talc and one or both of delaminated talc and perlite.

According to one embodiment of the present invention, the inorganic particulate material is delaminated.

In one embodiment, the skin care composition according to the present invention may be a water-in-oil emulsion, an oil-in-water emulsion, or an emulsified gel, and may be selected from a sun care composition, a day cream, a body lotion, a shower gel, a shampoo, a foundation, or a baby cream. It was found that the present invention was applicable to all these products, in particular if they provide UV protection.

In one embodiment of the present invention, the inorganic particulate material may be a talc having a lamellarity index of at least about 3.0, for example of at least about 4.0, and/or a D₅₀ of less than about 5.0 μm. It was found that such sun care compositions have further improved organoleptic characteristics while maintaining good SPF.

In one embodiment, the skin care composition according to the present invention comprises an inorganic particulate material which is optionally uncoated, such as a delaminated talc which is uncoated. It was found that such sun care compositions have improved organoleptic characteristics and, if required, maintain good SPF.

According to one embodiment of the present invention, the skin care composition may comprise from 0.1 to 10 wt.-% delaminated talc, such as for example at least about 2.0 wt.-% delaminated talc. For example, the skin care composition may comprise from 1 to 5 wt.-% delaminated talc, such as about 1 wt.-%, or about 2 wt.-%, or about 3 wt.-%, or about 4 wt.-%, or about 5 wt.-% delaminated talc. Such skin care compositions have been found to have good organoleptic characteristics while maintaining, if applicable, good SPF. In particular, such compositions have been found to have improved sensory properties with respect to their creaminess, reducing any powdery feel after application, improved optical properties, such as pearlescence or blurriness on the skin, or whiteness of the formulation, or they may even reduce phase separation and improve emulsion stability.

According to one embodiment of the present invention, the skin care composition may comprise no more than about 8.0 wt.-% TiO₂, and no selenium yeast complex. It was found that such skin care compositions have improved organoleptic characteristics with improved SPF, if applicable.

According to one embodiment of the present invention, the skin care composition may comprise a mineral UV-filter and/or an organic UV-filter. A mineral UV filter may be selected from at least one of ZnO and TiO₂. Such skin care compositions have been found to have good organoleptic characteristics with improved SPF.

In a further embodiment of the present invention, the skin care composition is substantially free of a mineral UV filter. It was found that such skin care compositions have improved organoleptic characteristics while maintaining good to satisfactory SPF.

In a further embodiment of the present invention, the skin care composition may further comprise one or more components selected from the group of emollients, emulsifiers, hydrants, thickeners and/or surfactants, and may be present in the form of a cream, or a cream-to-powder, or an ointment, or a lotion, or in a sprayable form. Such compositions have been found to have improved organoleptic properties while maintaining good SPF.

According to one embodiment of the present invention, the skin care composition may have an SPF of 6 or more.

In one embodiment, the present invention discloses a skin care composition which is a sun care composition comprising talc, wherein the talc is delaminated, and/or the talc has a BET surface area of least about 10.0 m²/g. According to some embodiments, the talc is also uncoated. A sun care composition according to this invention has improved organoleptic compositions and good SPF.

In one embodiment, the skin care composition according to the present invention comprises a delaminated talc which is optionally uncoated. In a further embodiment, the skin care composition of the present invention may comprise a talc with a surface area of at least about 10 m²/g which is optionally uncoated. It was found that such sun care compositions have improved organoleptic characteristics and good SPF, if required.

Also comprised in the present invention is the use of inorganic particulate materials in skin care compositions, wherein the inorganic particulate material is selected from the group consisting of delaminated talc, delaminated bentonite, an alkaline earth metal carbonate or sulphate (such as calcium carbonate, magnesium carbonate, dolomite, or gypsum), a hydrous kandite clay (such as delaminated kaolin, halloysite or ball clay), an anhydrous or calcined kandite clay (such as metakaolin or fully calcined kaolin, wollastonite, delaminated mica, perlite or diatomaceous earth), or magnesium hydroxide, or aluminium trihydrate, or combinations thereof, and/or wherein the inorganic particulate material has a BET surface area of at least about 10 m²/g. It was found that use of such inorganic particulate materials in a skin care composition improves the organoleptic properties while maintaining good SPF, if required.

According to a further aspect of the present invention, an inorganic particulate material, which may be a talc, a perlite or a diatomaceous earth, may be used as an extender for a mineral UV filter or an organic UV filter in a sun care composition, wherein the talc is a delaminated talc, and/or wherein the talc has a BET surface area of at least about 10 m²/g, and/or wherein the talc is uncoated. It was found that this use may improve the organoleptic properties while maintaining good SPF in a sun care composition.

According to one aspect of the present invention, an inorganic particulate material, which may be a talc, a perlite or a diatomaceous earth, may be used as a total replacement for a mineral and/or for an organic UV filter in a sun care composition, wherein the talc is a delaminated talc, and/or wherein the talc has a BET surface area of at least about 10 m²/g, and/or wherein the talc is uncoated. It was found that with such use, improved organoleptic characteristics may be obtained, while good SPF is achieved.

According to one aspect of the present invention, an inorganic particulate material which may be a talc, a perlite or a diatomaceous earth, wherein the talc is a delaminated talc, and/or wherein the talc has a BET surface area of at least about 10 m²/g, and/or wherein the talc is uncoated, may be used in order to improve the organoleptic properties of a sun care composition. It was found that this may be achieved by maintaining good SPF.

According to the present invention, the said organoleptic characteristics may include one or more of the properties selected from the group applicability, touch, softness, stickiness, and streaking, spreading, penetration, and combinations thereof.

According to one aspect of the present invention, an inorganic particulate material which may be a talc, a perlite or a diatomaceous earth, wherein the talc is a delaminated talc, and/or wherein the talc has a BET surface area of at least about 10 m²/g, and/or wherein the talc is uncoated, may be used for reducing the amount of mineral and/or organic UV filter in a sun care composition, such as for example at least a portion of mineral and/or organic UV filter in a sun care composition.

Also part of the present invention is a method of improving an organoleptic property of a skin care composition, for example of a sun care composition comprising mineral and/or organic UV filter, the method comprising incorporating an inorganic particulate material, such as talc, a perlite or a diatomaceous earth, in the sun care composition, wherein the talc is delaminated, and/or the talc has a BET surface area of least about 10 m²/g, and/or the talc is uncoated. According to one embodiment, the amount of any mineral and/or organic UV filter in the sun care composition is reduced or even removed in its entirety.

Also part of the present invention is a method of reducing the amount of mineral or organic UV filter in a sun care composition without adversely effecting the SPF of the sun care composition, the method comprising partially or totally replacing the mineral or organic UV filter with an inorganic particulate material, such as talc, a perlite or a diatomaceous earth, wherein the talc is delaminated, and/or the talc has a BET surface area of least about 10 m²/g, and/or the talc is uncoated.

The invention will be further illustrated by reference to Examples and the following detailed description. It is understood that the following description of the present invention and shall not be limiting the scope of the claims.

### DETAILED DESCRIPTION OF THE INVENTION

As used herein, “specific surface area (BET)” is understood to mean the area of the surface of the particles of the powder with respect to unit mass, determined according to the BET method by the quantity of argon adsorbed on the surface of the said particles so as to form a monomolecular layer completely covering the said surface (measurement according to the BET method, AFNOR standard X 11-621 and 622).

According to one embodiment, the inorganic material used herein can have a BET surface area of at least about 10 m²/g, such as for example at least about 15 m²/g, or at least about 20 m²/g.

As used herein, the term “delaminated talc” refers to a talc prepared according to a process for treating lamella-type minerals as described in U.S. Pat. No. 6,348,536 B1. Lamellar type minerals have a structure consisting of stacks of elementary leaves. During delamination they are treated to obtain a powder with high lamellarity. Similar processes are known to the skilled person for the provision of delaminated bentonite, delaminated mica, and delaminated kaolin, as used herein.

As used herein, the “lamellarity index” characterizes the shape of the particle, and more particularly its aspect ratio (large dimension/thickness). In all the following, this lamellarity index will be measured by the difference between, on the one hand, the value of the mean dimension of the particles of the powder obtained by a particle size measurement by Malvern laser diffraction D₅₀ las, obtained using a wet method (standard AFNOR NFX11-666) and, on the other hand, the value of the mean diameter D₅₀ sed obtained, by a measurement by sedimentation using a “Sedigraph” (standard AFNOR XII-683), this difference being related to the mean diameter D₅₀. Reference may be made to the article <<G. BAUDET and J. P. RONA, Ind. Min. Mines et Carr. Les techn. June, July 1990, pp 55-61>> which shows that this index is correlated to the mean ratio of the largest dimension of the particle to its smallest dimension.

As used herein, “high lamellarity” is understood to mean a powder of which the lamellarity index is high and in particular greater than 2.8. According to one embodiment, the talc used herein can have a lamellarity index of at least about 3.0, such as for example at least about 3.5, or at least about 4.0.

As used herein, “SPF” is the Sun Protection Factor, determined by in vitro measurements based on transmittance according to forthcoming ISO 24445 (pre-irradiation and spectrophotometer). As used herein, a satisfactory SPF may be an SPF of 4 or more, or an SPF of 6 or more, or an SPF of 8 or more, or an SPF of 10 or more.

As used herein, the terms “without adversely effecting the SPF” is understood to describe a variation in SPF which is either any increase of SPF, or a decrease of SPF of no more than 25% of an initial value.

The present invention according to the appended claims provides skin care compositions having improved organoleptic characteristics. It was found that skin care compositions according to the present invention may have improved organoleptic characteristics while maintaining, if applicable, good SPF. In particular, such compositions have been found to have improved sensory properties with respect to their creaminess, reducing any powdery feel after application, improved optical properties, such as pearlescence or blurriness on the skin, or whiteness of the formulation, or they may even reduce phase separation and improve emulsion stability. It was further found that inorganic particulate materials, such as for example talc powders, can be employed to complement, or extend, or partially or entirely replace mineral or organic UV filters, if present, in order to improve organoleptic characteristics while maintaining a good SPF value, if required. The inventors have surprisingly found that some inorganic particulate materials, such as some talc grades, improve the SPF and organoleptic characteristics of sun cream, providing that the talc is either delaminated, or the talc has a BET surface area of at least about 10 m²/g, or the talc is uncoated, or a combination of these properties.

Talc in itself does not absorb UV radiation and cannot be considered as a mineral UV filter in its own right. The same applies to all other inorganic particulate materials concerned by the present invention. Nevertheless, it has been found, for example, that certain talcs (e.g. delaminated talcs), in combination with UV organic filters, maintain Sun Protection Factor (SPF) high even in the absence of a UV mineral filter such as TiO₂. Without wanting to be bound by theory, it is thought that some of these unexpected good results might be due to some talc grades acting as extenders for organic UV filters. In fact, the lipophilic surfaces of talc might absorb or link to some chemical groups of organic UV filters. This may cause a better dispersion of the filters and finally a better UV protection in a sun care composition.

According to the present invention, it is therefore provided that inorganic particulate materials as described above, such as talcs, may be used in skin care compositions to partially or entirely replace mineral UV filters, in order to overcome the drawbacks associated with TiO₂ and ZnO. Talc particles, for example, do not cause the appearance of white streaks on the skin upon application. It was further found that additional organoleptic characteristics can be observed, including consistency, spreadability, penetration, and stickiness.

For example, talcs as described above may be used to partially or fully replace TiO₂ in existing sun care compositions. For example, a high lamellarity talc may replace 25 wt.-% or more of the TiO₂ component in a sun care composition, or 50 wt.-% or more of the TiO₂ component, or 75 wt.-% or more of the TiO₂ component, or 90 wt.-% or more of the TiO₂ component, or 100% of the TiO₂ component. Similarly, the talcs as described above may replace equivalent portions of ZnO components, or equivalent portions of any other mineral UV filters from existing sun care compositions. The obtained compositions may have an SPF of 6 or more, such as for example 15 or more, 30 or more, or 50 or more. According to other embodiments, the sun care composition SPF can for example range from 6 to 70, from 10 to 50, or from 15 to 50. In any case the SFP values shall not adversely affect the SPF compared to a composition comprising an equivalent amount of TiO₂, ZnO or other mineral UV filter.

It was found that replacement of TiO₂ by talcs as described above in existing sun care compositions may cause slight reduction of SPF values therein, but at the same time improve organoleptic characteristics thereof. The organoleptic characteristics concern the visual appearance and tactile sensation of a composition when applied to the human skin, such as for example ease of application (applicability), sensation on touching the skin, for example consistency, softness or stickiness, and the appearance of streaking as unwanted white lines on the skin after application.

### Examples

Several sun care compositions were provided, having similar compositions. The compositions comprised a base oil-in-water emulsion, comprising the following organic UV filters: butyl methoxydibenzoylmethane (1 wt.-%), PEG PABA (3 wt.-%), ethylhexyl methoxycinnamate (5 wt.-%). In addition, with the exception of the composition according to Comp. Ex. 1, the compositions each comprised 3 wt.-% of an inorganic material, as described in Table I:

TABLE I

| Example | Inorganic material |
|---|---|
| Comp. 1 | None |
| Comp. 2 | TiO₂ |
| Comp. 3 | A |
| Comp. 4 | B |
| Comp. 5 | C |
| Inv. 1 | D |
| Inv. 2 | E |

The composition according to Comp. Ex. 1 did not contain any inorganic UV filter extender. The composition according to Comp. Ex. 2 contained 3 wt.-% TiO₂ as an inorganic UV filter. Inorganic UV filters extenders A to E are described in Table II:

TABLE II

| UV Filter | | | | | Lamellarity |
|---|---|---|---|---|---|
| Extender | Material | D₅₀ las (μm) | D₅₀ sed (μm) | BET | index |
| A | Talc | 3.5 | 1.1 | 19.5 | 2.2 |
| B | Chlorite | 9.5 | 5.8 | 7.2 | 0.6 |
| C | Talc | 11.0 | 4.0 | 6.5 | 1.8 |
| D | Talc | 23.7 | 2.9 | 22.0 | 7.2 |
| E | Talc | 5.7 | 1.1 | 22.0 | 4.2 |

Talcs D and E are delaminated talcs having a lamellarity index [(D₅₀ las−D₅₀ sed)/D₅₀ sed] of 7.17 and 4.18, respectively, and are therefore considered high lamellarity talcs.

All sun care compositions prepared as above were analysed for SPF (UV) in vitro after formation (t=0) and after three months storage at 43° C. (for accelerated ageing; t=3 months). The results are shown in Table III:

TABLE III

| Example | SPF (t = 0) | SPF (t = 3 months) |
|---|---|---|
| Comp. 1 | 22 | 30.4 |
| Comp. 2 | 25.7 | 39.1 |
| Comp. 3 | 23 | 27.2 |
| Comp. 4 | 22.7 | 29.5 |
| Comp. 5 | 21.2 | 21.2 |
| Inv. 1 | 23.7 | 33.4 |
| Inv. 2 | 23 | 32.3 |

It is shown that, at formation, the inventive compositions have SPF values that are broadly equivalent to those of the Comparative Examples. Concerning Inv. Ex. 1 and 2, the SPF values obtained were better than those of Comp. Ex. 1, 3, 4 and 5, but not as good as the SPF value obtained for Comp. Ex. 2, the composition comprising TiO₂.

The organoleptic characteristics of the various compositions were also tested. To that effect, creams were tested by a panel of 10 people. The results are summarized in Table IV below.

TABLE IV

| | Penetration | No Sticky | No White traces | Softness |
|---|---|---|---|---|
| Comp 1 | ++ | + | ++ | + |
| Comp 2 | −− | − | − | − |
| Comp 3 | + | ++ | ++ | + |
| Comp 4 | + | ++ | + | + |
| Comp 5 | ++ | + | + | ++ |
| Inv 1 | ++ | + | + | ++ |
| Inv 2 | + | + | + | ++ |

In general, it was found that the compositions according to Inv. Ex. 1 and 2 provided improved applicability, a reduced sensation of oily touch, and a softer touch compared to Comp. Ex. 2.

The following numbered paragraphs define particular embodiments of the present invention:
1. A sun care composition comprising talc, wherein:
   (i) the talc is delaminated; and/or
   (ii) the talc has a BET surface area of least about 10.0 m²/g; and/or
   (iii) the talc has a lamellarity index of at least about 3.0; and/or
   (iv) the talc is uncoated.
2. A sun care composition according to paragraph 1, wherein the talc is delaminated talc and optionally uncoated.
3. A sun care composition according to paragraph 1, wherein the talc has a ET surface area of least about 10.0 m²/g and optionally uncoated.
4. A sun care composition according to any preceding paragraph, wherein the talc has a lamellarity index of at least about 4.0.
5. A sun care composition according to any preceding paragraph, wherein the talc has a D₅₀ of less than about 5.0 μm.
6. A sun care composition according to any preceding paragraph, wherein the composition comprises from about 0.1 wt.-% to about 10 wt.-% talc, based on the total weight of the sun care composition.
7. A sun care composition according to any preceding paragraph, wherein the composition comprises at least about 2.0 wt.-% talc.
8. A sun care composition according to any preceding paragraph, wherein the composition comprises a mineral UV filter and/or an organic UV filter.
9. A sun care composition according to paragraph 8, wherein the mineral UV filter is TiO₂ and/or ZnO.
10. A composition according to any preceding paragraph, wherein the composition comprises less than about 8.0 wt.-% TiO₂, and/or wherein the composition does not comprise a selenium yeast complex.
11. A sun care composition according to any one of paragraphs 1 to 8, wherein the composition is substantially free of a mineral UV filter.
12. A sun care composition according to any preceding paragraph, wherein the composition comprises one or more emollients, emulsifiers, hydrants, thickeners and/or surfactants.
13. A sun care composition according to any preceding paragraph, wherein the composition is a sun screen, for example, a cream, or ointment, or lotion, or in sprayable form.
14. A sun care composition according to any preceding paragraph having a SPF of at least about 6.0.
15. Use of talc in a sun care composition, wherein:
   (i) the talc is delaminated; and/or
   (ii) the talc has a BET surface area of least about 10.0 m²/g; and/or
   (iii) the talc has a lamellarity index of at least about 3.0; and/or
   (iv) the talc has a D₅₀ of less than about 5.0 μm; and/or
   (v) the talc is uncoated.
16. Use of talc as a mineral or organic UV filter extender in a sun care composition, wherein:
   (i) the talc is delaminated; and/or
   (ii) the talc has a BET surface area of least about 10.0 m²/g; and/or
   (iii) the talc has a D₅₀ of less than about 5.0 μm; and/or
   (iv) the talc has a lamellarity index of at least about 3.0; and/or
   (v) the talc is uncoated.
17. Use of talc in a sun care composition as a total replacement for mineral and/or organic UV filter, wherein:
   (i) the talc is delaminated; and/or
   (ii) the talc has a BET surface area of least about 10.0 m²/g; and/or
   (iii) the talc has a lamellarity index of at least about 3.0; and/or
   (iv) the talc has a D₅₀ of less than about 5.0 μm; and/or
   (v) the talc is uncoated.
18. Use of talc in a sun care composition for enhancing an organoleptic property of the sun care composition, wherein:
   (i) the talc is delaminated; and/or
   (ii) the talc has a BET surface area of least about 10.0 m²/g; and/or
   (iii) the talc has a D₅₀ of less than about 5.0 μm; and/or
   (iv) the talc has a lamellarity index of at least about 3.0; and/or
   (v) the talc is uncoated.
19. Use according to paragraph 18, wherein the organoleptic property is one or more of applicability, touch, for example, softness or stickiness, and streaking.
20. A method of reducing the amount of mineral and/or organic UV filter in a sun care composition, said method comprising replacing at least a portion of mineral and/or organic UV filter with talc, wherein:
   (i) the talc is delaminated; and/or
   (ii) the talc has a BET surface area of least about 10.0 m²/g; and/or
   (iii) the talc has a D₅₀ of less than about 5.0 μm; and/or
   (iv) the talc has a lamellarity index of at least about 3.0; and/or
   (v) the talc is uncoated.
21. A method of improving an organoleptic property of a sun care composition, for example, a sun care composition comprising mineral and/or organic UV filter, said method comprising incorporating talc in the sun care composition, wherein:
   (i) the talc is delaminated; and/or
   (ii) the talc has a BET surface area of least about 10.0 m²/g; and/or
   (iii) the talc has a D₅₀ of less than about 5.0 μm; and/or
   (iv) the talc has a lamellarity index of at least about 3.0; and/or
   (v) the talc is uncoated.
   optionally wherein the amount of any mineral and/or organic UV filter in the sun care composition is reduced.
22. A method of reducing the amount of mineral or organic UV filter in a sun care composition without adversely effecting the SPF of the sun care composition, said method comprising partially or totally replacing the mineral or organic UV filter with talc, wherein:
   (i) the talc is delaminated; and/or
   (ii) the talc has a BET surface area of least about 10.0 m²/g; and/or
   (iii) the talc has a D₅₀ of less than about 5.0 μm; and/or
   (iv) the talc has a lamellarity index of at least about 3.0; and/or
   (v) the talc is uncoated.
23. A skin care composition comprising one or more inorganic particulate materials, wherein:
   (i) the inorganic particulate material is selected from the group consisting of delaminated talc, delaminated bentonite, an alkaline earth metal carbonate or sulphate, such as calcium carbonate, magnesium carbonate, dolomite, gypsum, a hydrous kandite clay such as delaminated kaolin, halloysite or ball clay, an anhydrous or calcined kandite clay such as metakaolin or fully calcined kaolin, wollastonite, delaminated mica, perlite or diatomaceous earth, or magnesium hydroxide, or aluminium trihydrate, or combinations thereof; and/or
   (ii) the inorganic particulate material has a BET surface area of least about 10.0 m²/g.
24. A skin care composition according to paragraph 23, wherein the inorganic particulate material is selected from the group consisting of delaminated talc, or an alkaline earth metal carbonate or sulphate, such as calcium carbonate, magnesium carbonate, dolomite, gypsum, or halloysite, or ball clay, or metakaolin, or wollastonite, or perlite or diatomaceous earth, or magnesium hydroxide, or aluminium trihydrate, or combinations thereof.
25. A skin care composition according to paragraph 23 or 24, wherein the inorganic particulate material comprises one or more of delaminated talc, diatomaceous earth and perlite.
26. A skin care composition according to any one of paragraphs 23 to 25, wherein the inorganic particulate material is selected from one of delaminated talc, diatomaceous earth and perlite.
27. A skin care composition according to any one of paragraphs 23 to 26, which is a water-in-oil emulsion, an oil-in-water emulsion, or an emulsified gel, and which is selected from a sun care composition, a day cream, a body lotion, a shower gel, a shampoo, a foundation, or a baby cream.
28. A skin care composition according to any one of paragraphs 23 to 27, wherein the inorganic particulate material is a mixture of delaminated talc and either one or both of diatomaceous earth and perlite.
29. A skin care composition according to any one of paragraphs 23 to 28, wherein the inorganic particulate material is delaminated.

1. A skin care composition comprising one or more inorganic particulate materials, wherein:
the inorganic particulate material comprises delaminated talc, and
the inorganic particulate material has a BET surface area of least about 10.0 m²/g.

2. (canceled)

3. (canceled)

4. (canceled)

5. A skin care composition according to claim 1,
wherein the skin care composition is a water-in-oil emulsion, an oil-in-water emulsion, or an emulsified gel, and
wherein the skin care composition is selected from a sun care composition, a day cream, a body lotion, a shower gel, a shampoo, a foundation, or a baby cream.

6. A skin care composition according to claim 1, wherein the talc has a lamellarity index of at least about 3.0, or a D₅₀ of less than about 5.0 μm by Sedigraph.

7. A skin care composition according to claim 1, wherein the inorganic particulate material is uncoated.

8. A skin care composition according to claim 1, wherein the composition comprises from about 0.1 wt.-% to about 10 wt.-% delaminated talc, based on, the total weight of the sun care composition.

9. A skin care composition according to claim 1, wherein the composition comprises at least about 2.0 wt.-% delaminated talc.

10. A skin care composition according to claim 1, wherein the composition comprises a mineral UV filter or an organic UV filter.

11. A skin care composition according to claim 10, wherein the mineral UV filter includes TiO₂ or ZnO.

12. A skin care composition according to claim 1, wherein the composition comprises less than about 8.0 wt.-% TiO₂, or wherein the composition does not comprise a selenium yeast complex.

13. A skin care composition according to claim 1, wherein the composition is substantially free of a mineral UV filter.

14. A skin care composition according to claim 1, wherein the composition comprises one or more emollients, emulsifiers, hydrants, thickeners or surfactants.

15. A skin care composition according to claim 1, wherein the composition is a sun screen, for example, a cream, or ointment, or lotion, or in sprayable form.

16. A skin care composition according to claim 1 having a SPF of at least about 6.0.

17. Use of an inorganic particulate material in a skin care composition, wherein:
the inorganic particulate material comprises delaminated talc; and
the inorganic particulate material has a BET surface area of least about 10.0 m²/g.

18. Use according to claim 17, wherein the said inorganic particulate material comprises a talc having a lamellarity index of at least about 3.0, a perlite, a diatomaceous earth or a combination of any of these.

19. A skin care composition according to claim 1, further comprising delaminated bentonite, an alkaline earth metal carbonate or sulphate, such as calcium carbonate, magnesium carbonate, dolomite, gypsum, a hydrous kandite clay such as delaminated kaolin, halloysite or ball clay, an anhydrous or calcined kandite clay such as metakaolin or fully calcined kaolin, wollastonite, delaminated mica, perlite or diatomaceous earth, magnesium hydroxide, aluminium trihydrate, or combinations thereof.

20. A use according to claim 17, wherein the inorganic particulate material further comprises: delaminated bentonite, an alkaline earth metal carbonate or sulphate, such as calcium carbonate, magnesium carbonate, dolomite, gypsum, a hydrous kandite clay such as delaminated kaolin, halloysite or ball clay, an anhydrous or calcined kandite day such as metakaolin or fully calcined kaolin, wollastonite, delaminated mica, perlite or diatomaceous earth, magnesium hydroxide, aluminium trihydrate, or combinations thereof.
